(19) United States
(12) Patent Application Publication
(10) Pub. No.: US 2025/0138127 A1
(43) Pub. Date: May 1, 2025

(54) IMAGING PHANTOM ASSEMBLY AND IMAGE GENERATION METHOD

(71) Applicant: Shanghai United Imaging Healthcare Co., Ltd., Shanghai (CN)

(72) Inventors: Can Liao, Shanghai (CN); Xiangbin Fang, Shanghai (CN)

(21) Appl. No.: 18/926,434

(22) Filed: Oct. 25, 2024

(30) Foreign Application Priority Data
Oct. 25, 2023 (CN) 202311396065.8

(51) Int. Cl. G01R 33/58; G01R 33/34

(52) U.S. Cl. CPC G01R 33/58; G01R 33/34084

(57) ABSTRACT

An imaging phantom assembly includes a phantom body and one or more coils. At least one part of at least one of the one or more coils is arranged in the phantom body.

### CROSS-REFERENCE TO RELATED APPLICATIONS

This application claims priority to Chinese Patent Application No. 202311396065.8, entitled “IMAGING PHANTOM ASSEMBLY AND IMAGE GENERATION METHOD” and filed on Oct. 25, 2023, the entire contents of which are incorporated herein by reference.

### TECHNICAL FIELD

This disclosure relates to the field of imaging correction technologies, and in particular to an imaging phantom assembly and an image generation method.

### BACKGROUND

During research and development, design, and generation of a medical device (e.g., a magnetic resonance imaging (MRI) device), due to limitations of physical design and manufacturing processes, magnetic field inhomogeneity may be incorporated into a generated main magnetic field and gradient nonlinearity may be incorporated into a gradient field. This influence may further lead to introduction of geometric distortion into an imaging space of the MRI device, thereby leading to geometric distortion in an imaging image. Therefore, there is a need to use a phantom to correct the geometric distortion in the imaging space. During the correction of distortion characteristics of a large-sized imaging space, due to a large volume of a phantom required for geometric distortion, a manner in which a radio frequency receiving coil covers the phantom leads to a limitation on the quality of a signal in a central region of the phantom that can be acquired by the radio frequency receiving coil, and geometric distortion correction imaging with high resolution and a high signal-to-noise ratio cannot be realized.

### SUMMARY

In one aspect of this disclosure, an imaging phantom assembly is provided, including a phantom body and one or more coils. At least one part of at least one of the one or more coils is arranged in the phantom body.

In another aspect of this disclosure, an image generation method is provided, including: acquiring a magnetic resonance signal of an imaging phantom assembly under a radio frequency signal, the imaging phantom assembly including a phantom body and one or more coils, at least one part of at least one of the one or more coils being arranged in the phantom body; and generating an image based on the magnetic resonance signal.

### BRIEF DESCRIPTION OF THE DRAWINGS

This disclosure will be further described with exemplary embodiments. The exemplary embodiments will be described in detail with reference to the accompanying drawings. These embodiments are not limiting. In these embodiments, same numbers represent same structures.

FIG. 1 is a schematic diagram of simplified modules of an imaging phantom assembly according to some embodiments of this disclosure;

FIG. 2 is a schematic diagram of the imaging phantom assembly according to some embodiments of this disclosure;

FIG. 3 is a schematic diagram of a single-layer structure of the imaging phantom assembly according to some embodiments of this disclosure;

FIG. 4 is a schematic structural diagram of the imaging phantom assembly according to some other embodiments of this disclosure;

FIG. 5 is a schematic structural diagram of a second phantom layer according to some other embodiments of this disclosure;

FIG. 6 is a schematic structural diagram of the imaging phantom assembly according to some other embodiments of this disclosure, in which the imaging phantom assembly includes an insert layer; and

FIG. 7 is a schematic diagram of an imaging phantom assembly according to some other embodiments of this disclosure.

FIG. 8 is a schematic diagram of an imaging phantom assembly according to some other embodiments of this disclosure.

FIG. 9 is a schematic diagram of an imaging phantom assembly according to some other embodiments of this disclosure.

FIG. 10 is an exemplary flowchart of an image generation method according to some other embodiments of this disclosure.

### DETAILED DESCRIPTION

In order to describe the technical solutions of the embodiments of this disclosure more clearly, the accompanying drawings required to be used in the description of the embodiments will be briefly introduced below. Apparently, the accompanying drawings in the following description merely illustrate some examples or embodiments of this disclosure. For those of ordinary skill in the art, this disclosure can also be applied to other similar scenarios according to these accompanying drawings without creative efforts. Unless apparent from the context or unless otherwise stated, same reference numerals in the drawings denote a same structure or operation.

As shown in this disclosure and claims, words such as “a/an”, “one”, and/or “the” do not specifically refer to singular forms and may include plural forms unless the context clearly indicates an exception. Generally, the terms “comprise” and “include” merely imply inclusion of clearly identified steps and elements, and these steps and elements do not constitute an exclusive list. The method or device may also include other steps or elements.

During research and development, design, and generation of an MRI device, generally, due to limitations of physical design and manufacturing processes, magnetic field inhomogeneity and gradient nonlinearity are incorporated into a main magnetic field and a gradient field that are generated. This influence may lead to introduction of geometric distortion into an imaging space of MRI, thereby resulting in geometric distortion in an image, that is, deviation of coordinate points displayed in an MRI image from known positions or an incorrect proportion of a distance between any two points in the MRI image.

In some solutions, technologies for dealing with field inhomogeneity generally include collecting magnetic field intensity at each point in a spatially homogeneous field (e.g., a magnetic field space corresponding to an imaging space) by a magnetic field intensity measurement tool, and correcting magnetic field homogeneity by a ferromagnetic material such as a silicon steel sheet based on spatial distribution results of the magnetic field intensity. However, the homogeneous field correction method is relatively rough and difficult to meet geometric distortion correction requirements of high-resolution and high-precision MRI imaging such as radiotherapy. Moreover, during an actual operation, this solution can only compensate for homogeneity of a surface of the spatially homogeneous field (e.g., a surface of a spherically homogeneous field) relatively roughly. The accuracy of the actual collected magnetic field intensity at each point in the spatially homogeneous field is low, and compensation accuracy cannot meet the requirements. Therefore, in the case of higher requirements for imaging resolution and geometric accuracy, there is a need to place a phantom configured to correct geometric distortion (a surface of the phantom is covered with a radio frequency coil) in the spatially homogeneous field, image the phantom to obtain a corrected image, and present distortion characteristics in the spatially homogeneous field based on a geometric structure in the corrected image, thereby correcting the geometric distortion. In some solutions, when an imaging operation is performed on a large-sized phantom, types of coils configured to cover the phantom mainly include a volume transmit coil (VTC), a body array coil (BAC), and a spine coil (SPC), or a combination thereof. However, the actual imaging effect of the obtained corrected image is limited by a manner in which the radio frequency coil covers the phantom. During actual sampling, the quality of a signal in a central region of the phantom acquired by the radio frequency receiving coil is poor, making it difficult to identify and calculate geometric distortion. In order to solve the limitation on the actual imaging effect caused by the manner in which the radio frequency coil covers the phantom, a small-sized phantom may be used in some imaging solutions. However, the size of the spatially homogeneous field that can be corrected by this method is limited by the volume of the phantom. For example, when a spatially homogeneous field to be corrected has a larger size, the small-sized phantom cannot cover the entire spatially homogeneous field, so the geometric distortion cannot be corrected. If compensation is made by moving the phantom, more fusion errors between mechanical tail swing and the corrected image may be brought.

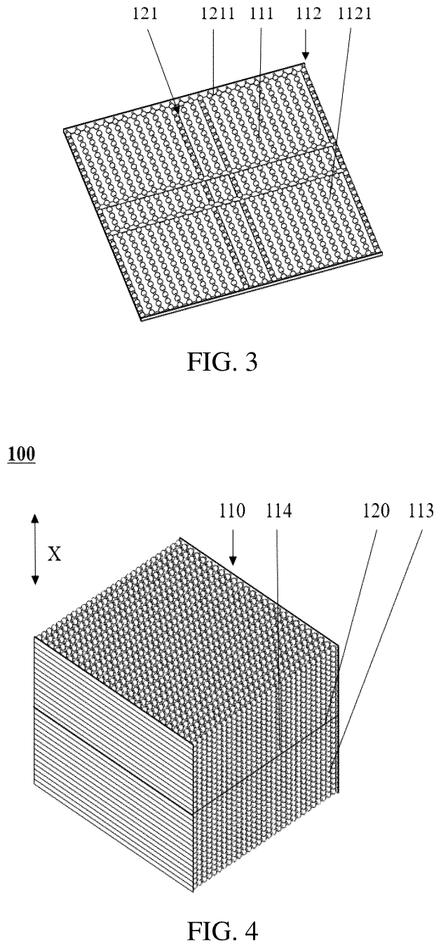

The imaging phantom assembly according to the embodiments of this disclosure applies to geometric distortion correction in spatially homogeneous fields in the medical and physical fields. Through the imaging phantom assembly according to this disclosure, signals from various parts of an imaging phantom acquired by the radio frequency coil can have high quality and accuracy, thereby obtaining a high-quality and high-accuracy magnetic resonance signal, so that an imaging effect of a finally generated corrected image for geometric distortion correction is no longer limited by the manner in which the radio frequency coil covers the phantom, thereby achieving geometric distortion correction imaging with high resolution and a high signal-to-noise ratio, and further improving a geometric distortion correction effect. In a specific application scenario, the imaging phantom assembly applies to geometric distortion correction of an MRI device. The imaging phantom assembly includes a phantom body and one or more coils. By arranging at least one part of the one or more coils in the phantom body, when geometric distortion correction imaging is performed on the phantom body, a maximum distance between the coil and various parts of the phantom body (e.g., the coil and a central region of the phantom body) is shorter, which improves the quality of a signal of the phantom body acquired by the coil, has higher accuracy, and can further improve the geometric distortion correction effect. The distance in one or more embodiments of this disclosure refers to a spacing in an imaging space of a to-be-corrected device (e.g., an MRI device) along a direction of a main magnetic field of the to-be-corrected device. For example, in the embodiments shown in FIG. 2, the direction of the main magnetic field of the MRI device may be represented by an arrow X. Therefore, the maximum distance between the coil and various parts of the phantom body in the direction of the arrow X when at least one part of the one or more coils are arranged in the phantom body is shorter than that when the coils are arranged at two ends of the phantom body along the direction X. In some embodiments, by designing the number of coils, relative positions of the coils and the phantom body, and parameters of the coils, different geometric distortion correction accuracy requirements can be met.

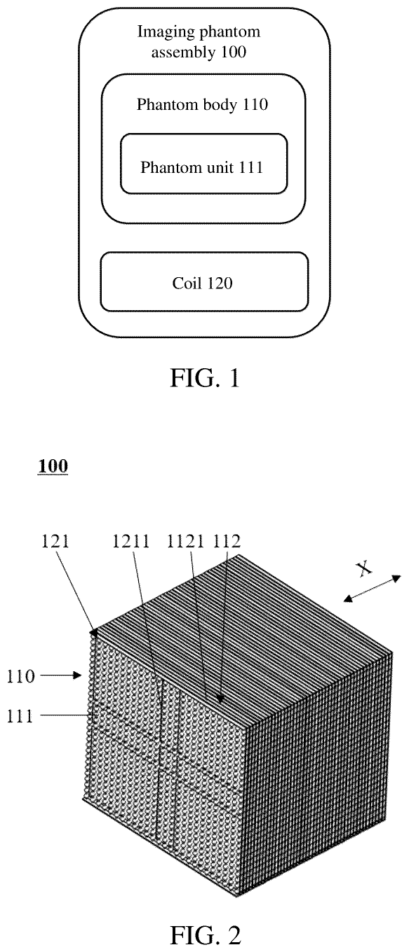

In some embodiments, as shown in FIG. 1, an imaging phantom assembly 100 includes a phantom body 110 and one or more coils 120, and at least one part of the one or more coils 120 are arranged in the phantom body 110. The phantom body 110 refers to an imaging phantom configured to correct geometric distortion of a to-be-corrected device.

In some embodiments, referring to FIG. 1 and FIG. 2, the phantom body 110 may include a plurality of phantom units 111. The phantom unit 111 may have a regular shape (e.g., a sphere, a cylinder, a cuboid, a cube, or the like) or an irregular shape. The plurality of phantom units 111 may be arranged in a specific manner. The shape of the phantom body 110 is related to an arrangement manner of the plurality of phantom units 111. For example, in the embodiments shown in FIG. 2, the plurality of phantom units 111 may form a phantom body 110 in a shape of a cuboid. In another example, the plurality of phantom units 111 may form a phantom body 110 in a shape of a cylinder. In yet another example, the plurality of phantom units 111 may form a phantom body 110 in a shape of a sphere.

In some embodiments, the phantom unit 111 includes any material capable of generating magnetic resonance under a magnetic field. In some embodiments, the phantom unit 111 may include a housing and a substance with which the housing is filled. The substance with which the housing is filled is a material capable of generating magnetic resonance under a magnetic field. Therefore, nuclei in the filling substance within the housing can enter a magnetic resonance state under the action of a magnetic field.

In some embodiments, the phantom unit 111 may include a body phantom, a water phantom, or the like. A difference between the body phantom and the water phantom includes different types of substances with which the phantoms are filled. For example, when the filling substance in the phantom unit 111 is a liquid substance such as water or salt liquid, the phantom unit 111 may be a water phantom. In another example, when the filling substance in the phantom unit 111 is a material with a density similar to that of human tissue (e.g., plastic, epoxy resin, polyurethane, Mix D, cork, human bone, artificial bone, a cast material, syntactic foam, rubber, foam rubber, isocyanate rubber, a thermoplastic material, Mix Dp, M3, an E-type material, and various other solids and liquids), the phantom unit 111 may be a body phantom. For ease of description, some embodiments of this disclosure are described based on an example in which the filling substance is water. When geometric distortion correction imaging is performed, after a radio frequency signal is fed into the coil 120, the coil 120 may generate a magnetic field and generate radio frequency energy, and hydrogen atoms in the phantom unit 111 (i.e., hydrogen atoms in water) may receive the radio frequency energy and enter an excited state (i.e., a magnetic resonance state). After feeding of the radio frequency signal is stopped, the hydrogen atoms in the excited state may radiate energy outward, and a radiation signal is captured by the coil 120, and then a magnetic resonance signal corresponding to each phantom unit 111 is obtained. After a series of processing on the magnetic resonance signal, a corrected image for geometric distortion correction imaging can be obtained. The process of the hydrogen atoms from entering the excited state to radiating the energy outward may be called relaxation, so the signal of the hydrogen atoms radiating the energy outward that is acquired by the coil 120 may also be called a relaxation signal. More details on geometric distortion correction imaging may be obtained with reference to FIG. 10 and the description of the embodiments thereof, which are not described herein again.

In some cases, since at least one part of the one or more coils 120 are arranged in the phantom body 110, i.e., at least one part of at least one of the one or more coils 120 is arranged in the phantom body 110, a maximum distance between the phantom unit 111 in a central region of the phantom body 110 and the coil 120 (e.g., the nearest coil) is shortened. The shortening of the distance enables an imaging effect of the corrected image to be no longer limited by the manner in which the coil 120 covers the phantom body 110 when geometric distortion correction imaging is performed on the phantom body 110, and quality and accuracy of a signal of the phantom unit 111 at each position acquired by the coil 120 are higher, and the corrected image obtained by correction imaging has a higher signal-to-noise ratio and higher resolution, thereby further improving the geometric distortion correction effect.

In some embodiments, the phantom body 110 may be an entirety, and at least one part of at least one of the one or more coils 120 may be arranged in the phantom body 110. For example only, the plurality of phantom units 111 forming the phantom body 110 are connected to one another and are inseparable, so that the phantom body 110 becomes an independent entirety, and the one or more coils 120 may be embedded in a gap between two adjacent phantom units 111, or the one or more coils 120 may be wound around one or more phantom units 111 to achieve a purpose of being arranged in the phantom body 110. In some embodiments, the one or more coils 120 may be completely arranged in the phantom body 110. For example, some coils 120 may be completely embedded in the phantom body 110, and other coils 120 may be arranged at at least one end of the phantom body 110 along a direction of a main magnetic field (as shown by the direction of the arrow X in FIG. 2). In some embodiments, one part of the one or more coils 120 may be completely arranged in the phantom body 110, and the other part may be arranged outside the phantom body 110. For example, one part of a coil 120 may be embedded in the phantom body 110, and the other part may be arranged at at least one end of the phantom body 110 along the direction of the main magnetic field.

In some embodiments, referring to FIG. 1 and FIG. 2, the phantom body 110 includes a plurality of phantom layers 112, the plurality of phantom layers 112 are stacked, the one or more coils 120 include one or more coil layers 121, and at least one of the one or more coil layers 121 is arranged between two adjacent phantom layers 112.

The plurality of phantom layers 112 are stacked, which means that the plurality of phantom layers 112 are stacked in sequence with a same placement posture or orientation. For example, in the embodiments shown in FIG. 2, it may be regarded as that the plurality of phantom layers 112 are stacked in parallel layer by layer along its thickness direction. In some embodiments, a stacking direction of the plurality of phantom layers 112 may be parallel to the direction of the main magnetic field, as shown by the direction of the arrow X in FIG. 2.

In this embodiment, since the plurality of phantom layers 112 are stacked, when at least one coil layer 121 is arranged between two adjacent phantom layers 112, the at least one coil layer 121 is arranged inside the imaging phantom assembly 100 (surfaces of the phantom layers 112 at two ends in the stacking direction may be regarded as surfaces of the imaging phantom assembly 100), which shortens a maximum distance between each phantom layer 112 and the coil layer 121, especially the distance between the phantom layer 112 in the central region of the phantom body 110 and the coil layer 121. In some cases, the shortening of the distance between the phantom layer 112 in the central region of the phantom body 110 and the coil layer 121 enables an imaging effect of the corrected image to be no longer limited by the manner in which the radio frequency coil covers the phantom when geometric distortion correction imaging is performed on the phantom body 110, and quality and accuracy of a signal of the phantom layer 112 at each position acquired by the coil layer 121 are higher, and the corrected image has a higher signal-to-noise ratio and higher resolution, thereby further improving the geometric distortion correction effect.

In some embodiments, the number of the phantom layer 112 may be adjusted according to a size of a to-be-correction imaging space. For example, when the size of the to-be-correction imaging space is larger, the number of the phantom layer 112 or a thickness of a single phantom layer 112 may be increased, to increase a size of the phantom body 110, thereby adapting to the imaging space of the larger size. When the size of the to-be-correction imaging space is smaller, the number of the phantom layer 112 or the thickness of the single phantom layer 112 may be decreased, to decrease the size of the phantom body 110, thereby adapting to the imaging space of the smaller size.

In some embodiments, when the phantom body 110 is formed by stacking a plurality of phantom layers 112, each phantom layer 112 may include a plurality of phantom units 111. The plurality of phantom units 111 may be arranged in a layered structure in a specific manner to form the phantom layer 112. For example, in the embodiments shown in FIG. 3, the plurality of phantom units 111 are distributed in a rectangular array to form a rectangular phantom layer 112, and a plurality of rectangular phantom layers 112 may be stacked to form a phantom body 110 in a shape of a cube or cuboid. In another example, the plurality of phantom units 111 are distributed in a circular matrix to form a circular phantom layer 112, and a plurality of circular phantom layers 112 may be stacked to form a phantom body 110 in a shape of a cylinder.

In some embodiments, as shown in FIG. 2 and FIG. 3, the phantom layer 112 may include a base 1121, the base 1121 is provided with a groove (not shown), and the plurality of phantom units 111 of each phantom layer 112 may be arranged in the groove of the base 1121 of the phantom layer 112. In some embodiments, the plurality of phantom units 111 in each phantom layer 112 are connected to each other. For example, in the embodiments shown in FIG. 3, the grooves in each row of the bases 1121 are connected in sequence, so the phantom units 111 in the grooves are also connected to each other.

In some embodiments, each phantom layer 112 may be formed by splicing two or more structures. For example, the phantom layer 112 may include a first splicing layer and a second splicing layer, the first splicing layer includes a plurality of hemispherical phantom units 111, and the second splicing layer also includes a plurality of hemispherical phantom units 111. When the first splicing layer and the second splicing layer are spliced into the phantom layer 112 along a stacking direction, the plurality of hemispherical phantom units 111 of the first splicing layer may be spliced with the plurality of hemispherical phantom units 111 of the second splicing layer to form a plurality of phantom units 111 in a shape of a sphere.

In some embodiments, one coil layer 121 may be provided, and in a stacking direction of the plurality of phantom layers 112, a ratio of distances between the coil layer 121 and two ends of the imaging phantom assembly 100 ranges from 4:6 to 6:4. That is, the coil layer 121 is positioned close to the central region of the phantom body 110 in the stacking direction, which can ensure that the distance between each phantom layer 112 and the coil layer 121 is shortened as much as possible while the number of the coil layer 121 is minimized, thereby improving quality and accuracy of the relaxation signal collected by the coil layer 121. For example only, a size of the phantom body 110 in the stacking direction is 500 mm (a size difference between the coil layer 121 and the phantom layer 112 in the stacking direction is large, so the influence of the coil layer 121 on the size of the phantom body 110 can be ignored). If the coil layer 121 is arranged at one end of the phantom body 110 in the stacking direction, a maximum distance between the coil layer 121 and each phantom layer 112 is 500 mm (that is, a distance between the coil layer 121 and the other end of the phantom body 110 in the stacking direction). When the coil layer 121 is positioned close to the central region of the phantom body 110 in the stacking direction, for example, distances from the coil layer 121 to the two ends of the phantom body 110 are 200 mm and 300 mm respectively, the maximum distance between the coil layer 121 and each phantom layer 112 is 300 mm. Therefore, the distance between the coil layer 121 and each phantom layer 112 will not be excessively long, which can ensure that the quality of the relaxation signal collected by the coil layer 121 meets the geometric distortion correction requirement.

In some embodiments, when one coil layer 121 is provided, the coil layer 121 may be arranged in the central region of the phantom body 110 in the stacking direction. In this case, the distances from the coil layer 121 to the two ends of the phantom body 110 in the stacking direction are the same, and the maximum distance between the coil layer 121 and each phantom layer 112 is smaller (approximately equal to half the size of the phantom body 110 in the stacking direction), which can further ensure that the quality of the relaxation signal collected by the coil layer 121 meets the geometric distortion correction requirement.

In some embodiments, a plurality of coil layers 121 may be provided, and the plurality of coil layers 121 may be spaced along a stacking direction of the plurality of phantom layers 112. Being spaced means that at least one phantom layer 112 is arranged between two adjacent coil layers 121. In some cases, by arranging a plurality of coil layers 121 at intervals between two ends of the phantom body 110 in the stacking direction, the maximum distance between the phantom layer 112 and the coil layer 121 can be further shortened, thereby more accurately collecting the relaxation signal of each phantom layer 112. In addition, due to the increase in the number of the coil layer 121, each coil layer 121 can acquire relaxation signals of a plurality of phantom layers 112, that is, each phantom layer 112 is sampled multiple times, thereby improving the quality and accuracy of a magnetic resonance signal finally generated and ultimately improving quality of the corrected image.

In some embodiments, the plurality of coil layers 121 may be distributed at equal intervals along the stacking direction of the plurality of phantom layers 112. Distribution at equal intervals means that spacings between all two adjacent coil layers 121 are equal. For example, one phantom layer 112 is arranged at intervals between each two adjacent coil layers 121. In some other embodiments, the plurality of coil layers 121 may be distributed at non-equal intervals along the stacking direction of the plurality of phantom layers 112. That is, spacings between all two adjacent coil layers 121 are not exactly the same.

In some embodiments, a plurality of coil layers 121 are provided, and in the stacking direction of the plurality of phantom layers 112, a ratio of distances between at least one coil layer 121 and two ends of the imaging phantom assembly 100 ranges from 4:6 to 6:4, so as to shorten distances between the phantom layers 112 at different positions in the stacking direction and the coil layer 121, so that the coil layer 121 can more accurately collect the relaxation signal of each phantom layer 112. In some embodiments, when a plurality of coil layers 121 are provided, one coil layer 121 may be arranged in the central region of the phantom body 110 in the stacking direction, so as to further shorten the distances between the phantom layers 112 at different positions in the stacking direction and the coil layers 121, so that the coil layer 121 can more accurately collect the relaxation signal of each phantom layer 112.

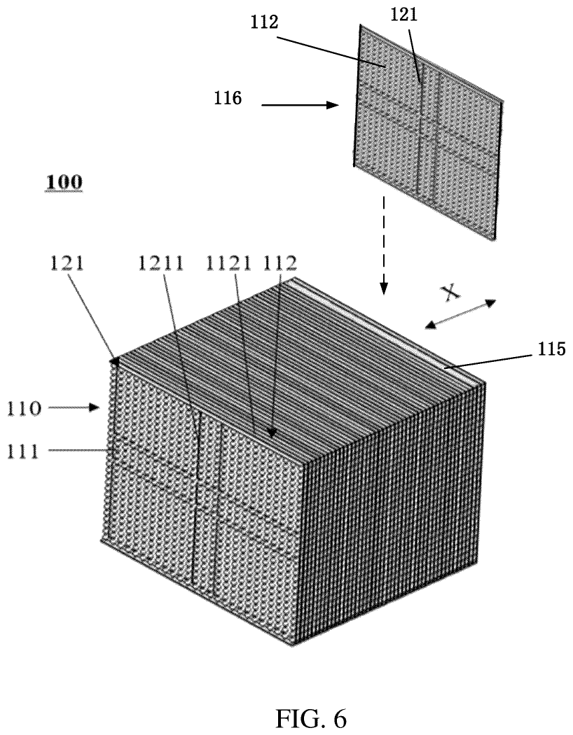

In some embodiments, referring to FIG. 6, a receiving groove 115 is formed between two adjacent phantom layers 112, and the coil layer 121 is detachably inserted into the receiving groove 115. In some cases, by arranging the coil layer 121 in the form of being inserted into the receiving groove 115, an operator can freely combine relative positions between the phantom layer 112 and the coil layer 121 and flexibly adjust the number and the position of the coil layer 121. When geometric distortion correction is performed on a large-sized imaging space, the imaging phantom assembly 100 includes a large number of phantom layers 112, and different geometric distortion correction accuracy requirements can be met by freely combining the relative positions between the phantom layer 112 and the coil layer 121, adjusting the number and the position of the coil layer 121, and flexibly adjusting the distance between the coil layer 121 and each phantom layer 112.

In some embodiments, the imaging phantom assembly 100 may further include an insert layer 116, the insert layer 116 includes the phantom layer 112 bonded with the coil layer 121, and the insert layer 116 is detachably inserted into the receiving groove 115. In some embodiments, the coil layer 121 may be bonded to the phantom layer 112 by adhesion, welding, snap connection, or the like. In some cases, by bonding the phantom layer 112 and the coil layer 121 into the insert layer 116, the coil layer 121 and the phantom layer 112 become an entirety, and the number of the coil layer 121 can be more conveniently increased or reduced, thereby reducing the complexity of mounting and removal of the coil layer 121. In addition, since the insert layer 116 is formed by bonding the phantom layer 112 and the coil layer 121, the phantom layer 112 of the insert layer 116 can support and fix the coil layer 121, and there is no need to provide an additional fixing structure between two adjacent phantom layers 112, which can effectively reduce design and manufacturing difficulty and costs and simplify a structure of the imaging phantom assembly 100.

In some embodiments, the coil layer 121 includes a subcoil 1211, and a projection of a region defined by the subcoil 1211 in the stacking direction covers a projection of the phantom layer 112 in the stacking direction, so that signals sent by the plurality of phantom units 111 of each phantom layer 112 during relaxation can be completely received by the coil layer 121, thereby obtaining a high-quality and high-accuracy magnetic resonance signal. For example, the subcoil 1211 encloses a region that forms a projection in the stacking direction, and the projection of the subcoil 1211 covers a projection of the phantom layer 112 in the stacking direction.

In some embodiments, the coil layer 121 may include a plurality of subcoils 1211, and the plurality of subcoils 1211 are not electrically communicated with each other. For example, in the embodiments shown in FIG. 3, the coil layer 121 includes four subcoils 1211. In some embodiments, a projection of regions defined by the plurality of subcoils 1211 in the stacking direction respectively corresponds to part of the projection of the phantom layer 112 in the stacking direction. In some cases, when the coil layer 121 includes a plurality of subcoils 1211, since each subcoil 1211 may simultaneously and independently collect relaxation signals of part of the phantom units 111 of each phantom layer 112 and then generate a corresponding magnetic resonance signal, the coil layer 121 can acquire the magnetic resonance signals more efficiently. In some other cases, since the number of phantom units 111 of the phantom layer 112 covered by each subcoil 1211 is smaller, a speed at which a single subcoil 1211 collects signals can be increased, thereby improving signal collection efficiency of the coil layer 121.

In some embodiments, the coil layer 121 includes a support apparatus (not shown), and the support apparatus is configured for the winding of the subcoils 1211 to form the coil layer 121. In some embodiments, the support apparatus may further provide a connection function for the phantom layers 112 on two sides of the coil layer 121. Exemplarily, the support apparatus may include a snap connection assembly, a clamping assembly, a support frame, or the like.

In some embodiments, the projection of the regions defined by the plurality of subcoils 1211 along the stacking direction covers the projection of the phantom layer 112 along the stacking direction, so that signals generated by relaxation of all the phantom units 111 of each phantom layer 112 can be completely received by the coil layer 121, thereby improving accuracy and quality of the relaxation signals. For example only, in the embodiments shown in FIG. 3, the projection of the phantom layer 112 along the stacking direction is in a shape of a rectangle, projections of four subcoils 1211 along the stacking direction are also in shapes of rectangles, and a combination of these rectangles can cover the projection of the phantom layer 112 along the stacking direction. However, it is not limited thereto. The projection of the phantom layer 112 along the stacking direction and/or the projection of the four subcoils 1211 along the stacking direction may alternatively be in any other shape, such as a trapezoid, a parallelogram, an ellipse, or a circle.

In some embodiments, in the plurality of subcoils 1211, no overlapping region exists between a region defined by one subcoil 1211 and a region defined by at least another subcoil 1211. For example, the coil layer 121 includes four subcoils 1211, a region defined by each subcoil 1211 is in a shape of a square, the four subcoils 1211 are arranged in a “grid” shape, and the projection thereof along the stacking direction just covers the projection of the phantom layer 112 along the stacking direction.

In some embodiments, in the plurality of subcoils 1211, an overlapping region exists between a region defined by one subcoil 1211 and a region defined by at least another subcoil 1211. For example only, in the embodiments shown in FIG. 3, the coil layer 121 includes four subcoils 1211 in shapes of rectangles, and an overlapping region exists between a region defined by each subcoil 1211 and regions defined by the other three subcoils 1211. However, it is not limited thereto. As required, the coil layer 121 may alternatively include other numbers and/or other shapes of subcoils 1211, and/or an overlapping region exists between regions defined by one part of the subcoils 1211 and regions defined by one or more other subcoils 1211.

In some cases, when an overlapping region exists between a region defined by one subcoil 1211 and a region defined by at least another subcoil 1211, it can be ensured that the corresponding phantom unit 111 near an edge of the region defined by each subcoil 1211 can be covered by the subcoils 1211 to prevent generation of an inaccurate magnetic resonance signal due to missing of the signal of the phantom unit 111 corresponding to the edge. Since the plurality of subcoils 1211 of each coil layer 121 are not communicated with each other, there may be no coupling problem in overlapping portions between one subcoil 1211 and the other subcoils 1211, which can effectively prevent interference with the signals acquired by the subcoils 1211. In addition, when an overlapping region exists between the regions defined by the plurality of subcoils 1211, the plurality of subcoils 1211 may repeatedly collect relaxation signals of the phantom units 111 in the overlapping region. Therefore, quality and accuracy of the relaxation signals of the phantom units 111 can be improved, thereby improving quality of the magnetic resonance signal.

In some other embodiments, the shape of the region defined by the subcoil 1211 may not be limited to the rectangle in FIG. 3. For example, the shape of the region defined may further include regular or irregular shapes such as a circle, a triangle, a polygon, or a rectangle. In an embodiment, the shapes of the regions defined by the plurality of subcoils 1211 may be the same. As shown in FIG. 3, the shapes of the regions defined by the four subcoils 1211 are all rectangles. In some embodiments, the shapes of the regions defined by the plurality of subcoils 1211 may be different. In some embodiments, the shape of the region defined may be adjusted according to the shape of the projection of the phantom layer 112 in the stacking direction, to ensure that a projection of a region defined by the coil layer 121 in the stacking direction can cover the projection of the phantom layer 112 in the stacking direction. For example, when the shape of the projection of the phantom layer 112 in the stacking direction is a square, to enable the plurality of subcoils 1211 to better cover the phantom layer 112, the shape of the region defined by the subcoil 1211 may be a square or a rectangle. In another example, when the shape of the projection of the phantom layer 112 in the stacking direction is a triangle, the shape of the region defined by the subcoil 1211 may be a triangle.

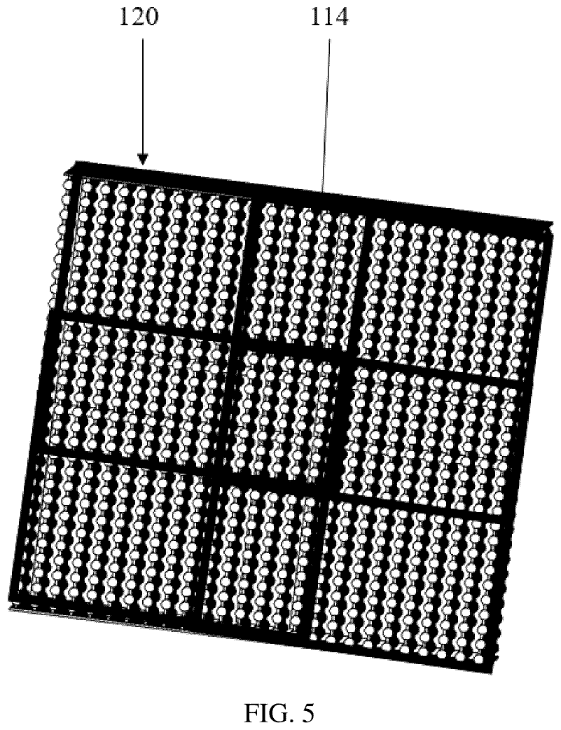

In some embodiments, as shown in FIG. 4 and FIG. 5, the phantom body 110 of the imaging phantom assembly 100 includes a plurality of first phantom layers 113 and one or more second phantom layers 114, the plurality of first phantom layers 113 are stacked, at least one of the one or more second phantom layers 114 are arranged between two adjacent first phantom layers 113, and the coil 120 is wound around the second phantom layer 114.

The first phantom layer 113 in this embodiment is the same as or similar to the phantom layer 112 in the foregoing embodiments. The second phantom layer 114 in this embodiment is different from the phantom layer 112 in the foregoing embodiments in that the coil 120 is wound around the second phantom layer 114. That is, the second phantom layer 114 and the coil 120 are integrated into a whole by winding. The second phantom layer 114 is detachably arranged between adjacent first phantom layers 113 instead of the coil 120 being detachably arranged between adjacent phantom layers, which can also easily increase or decrease the number of the coil 120, thereby reducing complexity of mounting and removal of the coil layer 121.

In the above embodiments, the phantom body is composed of multiple phantom layers, with coil layers arranged between the phantom layers or on specific phantom layers, such that at least a part of the coils is arranged in the phantom body.

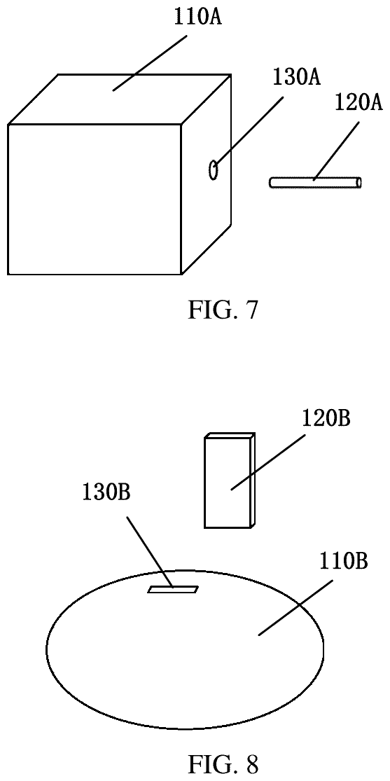

In some other embodiments, the phantom body and the coil can be configured using alternative structural arrangements. For example, the phantom body can be a single structure rather than a layered one, such as a single cubic phantom body 110A shown in FIG. 7 or a single elliptical phantom body 110B shown in FIG. 8. It is understood that the phantom body can also be of other regular or irregular structures. The phantom body 110A, 110B has a receiving groove 130A, 130B formed therein. The coil can be configured in different structures, such as a rod-shaped coil 120A shown in FIG. 7 or a strip-shaped coil 120B shown in FIG. 8, and the coil 120A, 120B can be detachably inserted into the receiving groove 130A, 130B. It is understood that layered coils also applies to the single phantom body 110A, 110B. As such, the distance between various parts of the phantom body and the coil can also be reduced.

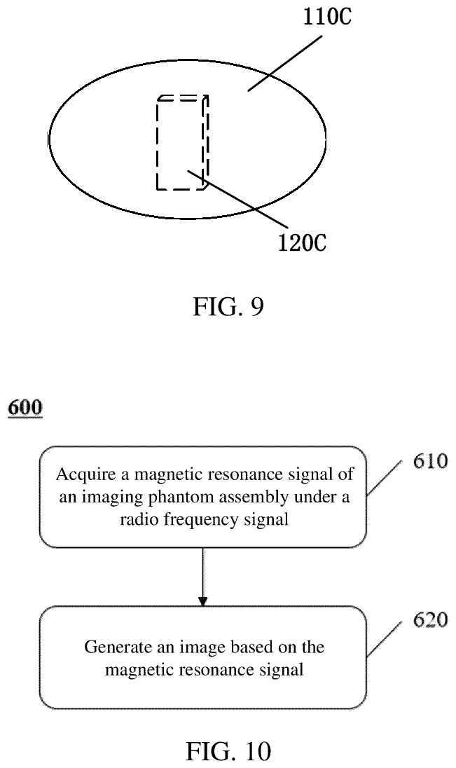

In another embodiment shown in FIG. 9, a coil 120C is formed in a phantom body 110C, which similarly reduces the distance between various parts of the phantom body 110C and the coil 120C. For instance, during the formation of the phantom body 110C, the coil 120C is placed directly within it and thus cannot be separated from the phantom body 110C.

In FIGS. 7-9, although only one coil is illustrated, it is understood that these embodiments may include one or more coils.

As shown in FIG. 10, this disclosure further provides an image generation method 600. The image generation method 600 may perform geometric distortion correction on a specific space of an MRI device based on the imaging phantom assembly described in other embodiments of this disclosure (e.g., the imaging phantom assembly 100 in FIG. 1 to FIG. 6). In some specific application scenarios, the image generation method 600 may perform geometric distortion correction on an imaging space of the MRI device. The image generation method 600 will be further introduced below based on an example in which the image generation method 600 is applied to geometric distortion correction on the imaging space of the MRI device. In some embodiments, the image generation method 600 may be performed manually by an operator or by a processor (not shown) of the MRI device. The image generation method 600 includes the following steps.

In step 610, a magnetic resonance signal of an imaging phantom assembly under a radio frequency signal is acquired.

Acquiring a magnetic resonance signal of an imaging phantom assembly under a radio frequency signal means that a magnetic resonance signal of a phantom unit of a phantom body (e.g., the phantom body 110 in FIG. 1 to FIG. 6) under the radio frequency signal is acquired. In some embodiments, before a magnetic resonance signal of the phantom body under the radio frequency signal is acquired, the imaging phantom assembly may be placed in an imaging space of a to-be-corrected device (e.g., the MRI device), and a plane of the coil is perpendicular to a direction of a main magnetic field of the MRI device, so that a direction of a magnetic field generated by the coil after receiving the radio frequency signal is parallel to the direction of the main magnetic field, improving quality and accuracy of the signal acquired by the coil. In some embodiments, the MRI device may include a moving device (not shown). The moving device may be connected to the imaging phantom assembly. The processor may drive the moving device to move the imaging phantom assembly to the imaging space of the MRI device, and the plane of the coil is perpendicular to the direction of the main magnetic field of the MRI device.

In some embodiments, a size of the imaging phantom assembly may be adjusted according to a size of the imaging space, to meet a correction requirement. In some embodiments, when the phantom body is an entirety (e.g., the phantom body 110 is directly formed by stacking a plurality of phantom units 111), the size of the phantom body may be adjusted by increasing or decreasing the number of the phantom units. In some embodiments, when the phantom body is formed by stacking a plurality of phantom layers, the size of the phantom body may be adjusted by increasing or decreasing the number of the phantom layers. In some embodiments, the number of the coils and relative positions between the coils and the phantom body may be adjusted according to a correction accuracy requirement. For example, when the correction accuracy requirement is high, the number of the coils may be increased. When the correction accuracy requirement is low, the number of the coils may be appropriately reduced. In another example, when the correction accuracy requirement is high, the coil may be arranged close to the central region of the phantom body.

In some embodiments, after the imaging phantom assembly is placed in the imaging space, a radio frequency signal transmitting assembly (not shown) of the MRI device is controlled to transmit a radio frequency signal to the coil of the imaging phantom assembly. After the radio frequency signal is fed into the coil, the coil may generate a magnetic field and generate radio frequency energy. Hydrogen atoms in the phantom unit of the phantom body receive the radio frequency energy and enter an excited state (i.e., a magnetic resonance state). In this case, the radio frequency signal transmitting assembly is controlled to stop operating, hydrogen atoms in the plurality of phantom units relax, and the coil captures relaxation signals of the hydrogen atoms and then generates a corresponding magnetic resonance signal.

In some embodiments, a plurality of coils are provided, the plurality of coils are arranged at intervals relative to the phantom body along the direction of the main magnetic field (e.g., the direction of the main magnetic field of the MRI device), and acquiring the magnetic resonance signal of the imaging phantom assembly under the radio frequency signal may include acquiring a plurality of subsignals, each of the plurality of subsignals being acquired by one coil, and determining the magnetic resonance signal based on the plurality of subsignals. In this embodiment, when the imaging phantom assembly includes a plurality of coils, each coil may independently collect the relaxation signal when the phantom unit radiates energy corresponding to each position of the phantom body along the direction of the main magnetic field, i.e., one coil may collect a plurality of relaxation signals when the phantom units at various positions of the phantom body along the direction of the main magnetic field radiate energy. The plurality of relaxation signals collected by each coil may be calculated and processed to obtain a subsignal. In some embodiments, the plurality of relaxation signals collected by one coil may be calculated and processed by summing, averaging, variance, or the like, to obtain a subsignal corresponding to the coil. Since each coil is arranged at a different position, each coil receives different radiation energy from the phantom units at various positions along the direction of the main magnetic field of the phantom body. Therefore, the plurality of relaxation signals collected by different coils are also different. For example, if the phantom unit at a certain position of the phantom body is farther away from the coil along the direction of the main magnetic field, the radiation energy received by the coil from the phantom unit is weaker, and the relaxation signal of the phantom unit that the coil can collect has a weaker intensity. If the phantom unit at a certain position of the phantom body is closer to the coil along the direction of the main magnetic field, the radiation energy received by the coil from the phantom unit is stronger, and the relaxation signal of the phantom unit that the coil can collect has a stronger intensity. Therefore, when the coils are arranged at different positions, the subsignals acquired by the coils are also different. Therefore, when the imaging phantom assembly includes a plurality of coils, each coil may acquire a different subsignal. For example only, the phantom body includes a plurality of phantom layers, one or more coils include one or more coil layers, at least one of the one or more coil layers is arranged between two adjacent phantom layers, and each coil layer may independently collect corresponding relaxation signals when the phantom layers radiate energy. That is, one coil layer may collect a plurality of relaxation signals when the phantom layers radiate energy. The plurality of relaxation signals collected by each coil layer may be calculated and processed to obtain a subsignal. In some embodiments, the magnetic resonance signal can be obtained by processing the plurality of subsignals acquired by the plurality of coils in a manner such as weighting. In some cases, when the imaging phantom assembly includes a plurality of coils, the plurality of coils may repeatedly collect relaxation signals when one or more phantom units at a same position of the phantom body along the direction of the main magnetic field (e.g., a plurality of phantom units forming a same phantom layer may be approximately regarded as being located at a same position in the direction of the main magnetic field) radiate energy, i.e., the one or more phantom units at the same position of the phantom body along the direction of the main magnetic field are sampled multiple times, thereby improving quality and accuracy of the magnetic resonance signal finally generated.

In some embodiments, when the imaging phantom assembly includes a plurality of coils, the radio frequency signal transmitting assembly may be controlled to feed radio frequency signals to the plurality of coils at the same time. In this case, the plurality of coils may generate magnetic fields and emit radio frequency energy at the same time and acquire subsignals of the phantom units at various positions along the direction of the main magnetic field of the phantom body approximately simultaneously, thereby improving image processing efficiency. In some embodiments, when the imaging phantom assembly includes a plurality of coils, the radio frequency signal transmitting assembly may be controlled to sequentially feed the radio frequency signals to each coil, and sequentially collect a plurality of subsignals acquired by the plurality of coils. Since no other coils collect relaxation signals and acquire subsignals at the same time when each coil collects relaxation signals and acquires subsignals, interference with collection of the relaxation signals and acquisition of the subsignals by the coil can be prevented, which can effectively avoid the problem of low quality of a magnetic resonance signal generated due to coupling of the plurality of coils. For example only, the imaging phantom assembly includes a first coil, a second coil, and a third coil. A radio frequency signal may be fed into the first coil first, and through the first coil, a plurality of first relaxation signals are collected and a first subsignal is acquired. Then, the same radio frequency signal is fed into the second coil, and through the second coil, a plurality of second relaxation signals are collected and a second subsignal is acquired. Finally, the same radio frequency signal is fed into the third coil, and through the third coil, a plurality of third relaxation signals are collected and a third subsignal is acquired. Finally, the magnetic resonance signal is determined based on the three subsignals.

In step 620, an image is generated based on the magnetic resonance signal.

In some embodiments, after the magnetic resonance signal of the imaging phantom assembly is acquired, the magnetic resonance signal may be processed, thereby generating the image. For example, K-space signal filling and signal post-processing may be performed on the magnetic resonance signal to obtain the image (i.e., a corrected image).

Based on different embodiments, beneficial effects of the imaging phantom assembly and the image generation method according to this disclosure include, but are not limited to, the following. (1) At least one of the one or more coils is arranged in the phantom body, so that the relaxation signals generated by the phantom layers at various positions in the phantom body can be well received by the coil, especially when the coil is closer to one or more phantom units in the central region of the phantom body, the received relaxation signals corresponding to one or more phantom units are more accurate, and the generated magnetic resonance signal has higher quality, thereby meeting the geometric distortion correction requirement. (2) By arranging a plurality of coil layers at intervals between two ends of the phantom body, it can be further ensured that the distance between the phantom layer at each position and the coil layer may not be excessively long, so that the signal corresponding to each phantom layer can be acquired more accurately. In addition, since the number of the coil layer increases and each coil can acquire signals of the plurality of phantom layers, the signals collected by the plurality of coil layers have cross data, which is equivalent to improving quality and accuracy of the received signals of the phantom layers, thereby further improving quality and accuracy of the magnetic resonance signal and ultimately improving imaging quality. (3) By arranging the coil layer in a form of being inserted into the receiving groove, the operator can freely combine the relative positions between the phantom layer and the coil layer, and when geometric distortion correction is performed on a large-sized imaging space, the imaging phantom assembly includes a large number of phantom layers, and the maximum distance between the coil layer and each phantom layer can be flexibly adjusted by freely combining the relative positions between the phantom layer and the coil layer and adjusting the number of the coil layer, so as to meet different geometric distortion correction accuracy requirements. (4) By integrating the phantom layer and the coil layer into an insert layer, the number of the coil layer can be more conveniently increased or decreased, thereby reducing complexity of mounting and removal of the coil layer. In addition, since the insert layer is formed by bonding the phantom layer and the coil layer, the phantom layer of the insert layer can support and fix the coil layer, and there is no need to provide an additional fixing structure between two adjacent phantom layers, which can effectively reduce design and manufacturing difficulty and costs and simplify a structure of the imaging phantom assembly. (5) When the coil layer includes a plurality of subcoils, since each subcoil can simultaneously and independently receive radiated energy of part of phantoms of the phantom layer and then generate a corresponding signal, the coil layer can collect signals more efficiently.

The above are only preferred embodiments of this disclosure and are not intended to limit this disclosure. Any modification, equivalent replacement, improvement, and the like made within the spirit and principles of this disclosure shall fall within the protection scope of this disclosure.

What is claimed is:

1. An imaging phantom assembly, comprising:
a phantom body; and
one or more coils, at least one part of at least one of the one or more coils being arranged in the phantom body.

2. The imaging phantom assembly according to claim 1, wherein the phantom body comprises a plurality of phantom layers, and the one or more coils comprise one or more coil layers, at least one of the one or more coil layers being arranged between two adjacent phantom layers.

3. The imaging phantom assembly according to claim 2, wherein the one or more coils comprise a plurality of coil layers, the plurality of coil layers being spaced along a stacking direction of the plurality of phantom layers.

4. The imaging phantom assembly according to claim 2, wherein the one or more coils comprise one coil layer, and in a stacking direction of the plurality of phantom layers, a ratio of distances between the coil layer and two ends of the phantom body ranges from 4:6 to 6:4.

5. The imaging phantom assembly according to claim 2, wherein a receiving groove is formed between two adjacent phantom layers, and the receiving groove are configured for receiving the one or more coil layers.

6. The imaging phantom assembly according to claim 5, wherein the one or more coil layers are detachably inserted into the receiving groove.

7. The imaging phantom assembly according to claim 5, wherein the imaging phantom assembly further comprises an insert layer, the insert layer comprising the phantom layer bonded with the one or more coil layers, and the insert layer being able to be detachably inserted into the receiving groove.

8. The imaging phantom assembly according to claim 2, wherein at least one of the one or more coil layers comprises a plurality of subcoils.

9. The imaging phantom assembly according to claim 8, wherein an overlapping region exists between a region defined by one of the subcoils of the coil layer and a region defined by at least another of the subcoils.

10. The imaging phantom assembly according to claim 2, wherein at least one of the one or more coil layers comprises a plurality of subcoils, wherein no overlapping region exists between a region defined by one of the subcoils and a region defined by at least another of the subcoils.

11. The imaging phantom assembly according to claim 10, wherein a projection of regions defined by the plurality of subcoils along a stacking direction covers a projection of the phantom layers along the stacking direction.

12. The imaging phantom assembly according to claim 1, wherein the phantom body comprises a plurality of first phantom layers and one or more second phantom layers, and at least one of the one or more second phantom layers being arranged between two adjacent first phantom layers; and
wherein the coil is wound around the second phantom layer.

13. The imaging phantom assembly according to claim 1, wherein a receiving groove is formed in the phantom, and the one or more coils are detachably inserted into the receiving groove.

14. The imaging phantom assembly according to claim 1, wherein the one or more coils are formed in the phantom body.

15. An image generation method, comprising:
acquiring a magnetic resonance signal of an imaging phantom assembly under a radio frequency signal, the imaging phantom assembly comprising a phantom body and one or more coils, at least one part of at least one of the one or more coils being arranged in the phantom body; and
generating an image based on the magnetic resonance signal.

16. The image generation method according to claim 15, wherein the one or more coils comprise a plurality of coils, and acquiring the magnetic resonance signal of the imaging phantom assembly under the radio frequency signal comprises:
controlling a radio frequency signal transmitting assembly to feed the radio frequency signal into the plurality of coils at the same time.

17. The image generation method according to claim 15, wherein the one or more coils comprise a plurality of coils, and acquiring the magnetic resonance signal of the imaging phantom assembly under the radio frequency signal comprises:
controlling the radio frequency signal transmitting assembly to sequentially feed the radio frequency signal to each of the coils.

18. The image generation method according to claim 15, wherein the phantom body comprises a plurality of phantom layers, and the one or more coils comprise one or more coil layers, at least one of the one or more coil layers being arranged between two adjacent phantom layers.

19. The image generation method according to claim 18, wherein a receiving groove is formed between two adjacent phantom layers, and the one or more coil layers are detachably inserted into the receiving groove.

20. The image generation method according to claim 18, wherein at least one of the one or more coil layers comprises a plurality of subcoils, and a region defined by each of the subcoils is in a shape of any one of a rectangle, a circle, a triangle, or a polygon.
